(19) United States
(12) Patent Application Publication
(10) Pub. No.: US 2013/0072514 A1
(43) Pub. Date: Mar. 21, 2013

(54) Sustained-Release Opioid Formulations and Methods of Use

(71) Applicants: Garth Boehm, Bridgewater, NJ (US); Alfred Liang, Bridgewater, NJ (US)

(72) Inventors: Garth Boehm, Bridgewater, NJ (US); Alfred Liang, Bridgewater, NJ (US)

(73) Assignee: Alpharma Pharmaceuticals, LLC, Bridgewater, NJ (US)

(21) Appl. No.: 13/413,334

(22) Filed: Mar. 6, 2012

Related U.S. Application Data
(63) Continuation of application No. 12/418,806, filed on Apr. 6, 2009, now abandoned.
Application No. 12/418,806 is a division of application No. 10/667,570, filed on Sep. 22, 2003, now abandoned.

(51) Int. Cl. A61K 31/485; A61P 25/04

(52) U.S. Cl. 514/282

(57) ABSTRACT

The invention combines two different subunits with different release profiles in novel sustained-release oral dosage forms. In particular, the oral dosage forms include a subunit that comprises an opioid analgesic and a sustained-release material, wherein the dissolution rate in-vitro of the subunit, when measured by the standard USP Drug Release test of U.S. Pharmacopeia XXVI (2003) <724>, is less than about 10% within about 6 hours and at least about 60% within about 24 hours; less than about 10% within about 8 hours and at least about 60% within about 24 hours; or less than about 10% within about 12 hours and at least about 60% within about 24 hours; the dosage form providing a duration of therapeutic effect of about 24 hours.

### FIELD OF THE INVENTION

The invention relates to an oral dosage form comprising an analgesic drug, particularly an opioid analgesic, in sustained-release form and a method of use, e.g., to treat pain.

### BACKGROUND OF THE INVENTION

It is the intent of all sustained-release preparations to provide a longer period of pharmacological response after the administration of the drug than is ordinarily experienced after the administration of the rapid-release dosage forms. Such longer periods of response provide for many inherent therapeutic benefits that are not achieved with corresponding short-acting, immediate-release preparations. This is especially true in the treatment of cancer patients, or other patients in need of treatment, for the alleviation of moderate to severe pain, where blood levels of an opioid analgesic medicament must be maintained at a therapeutically effective level to provide pain relief. Unless conventional, rapid-acting drug therapy is carefully administered at frequent intervals to maintain effective steady-state blood levels of the drug, peaks and valleys in the blood level of the active drug occur because of the rapid absorption and systemic excretion of the compound and through metabolic inactivation, thereby producing special problems in the maintenance of analgesic efficacy.

Many of the currently available oral opioid analgesic formulations must be administered every four to six hours; a selected few are formulated for less frequent 12-hour dosing and even fewer for 24-hour dosing, such as Kadian®, which is available in the United States from Faulding Laboratories, Inc. (Piscataway, N.J.).

Despite the availability of sustained-release formulations of opioids, none provide the optimum therapeutic effect because none maintain the blood concentration of the opioid at a constant or substantially constant level for 24 hours. All of these products provide opioid concentrations that vary with time, i.e., at certain points there are higher concentrations of the opioid than at other times. This means that at certain points in the 24 hour period, the patient may receive therapeutically effective amounts of the opioid, while at other points, the opioid in the blood may fall below therapeutic levels (i.e., pain relief may not be maintained).

Therefore, there exists a need for a sustained-release opioid formulation that provides a blood level of opioid to the patient at a substantially constant or therapeutically consistent level for about 24 hours. These and other advantages of the invention, as well as additional inventive features, will be apparent from the description of the invention provided herein.

### BRIEF SUMMARY OF THE INVENTION

The invention provides for a sustained-release oral dosage form that includes a subunit comprising an opioid analgesic and a sustained-release material, wherein the dissolution rate in-vitro of the subunit, when measured by standard USP Drug Release test of U.S. Pharmacopeia XXVI (2003) <724>, is less than about 10% within about 6 hours and at least about 60% within about 24 hours, preferably having a maximum rate of release from about 10% to about 50% per hour, more preferably from about 10% to about 25% per hour. In certain embodiments, the release rate in-vitro is preferably less than about 10% within about 8 hours and at least about 60% within about 24 hours, preferably having a maximum rate of release from about 10% to about 50% per hour, more preferably from about 10% to about 25% per hour. In other embodiments, the release rate in-vitro is preferably less than about 10% within about 10 hours and at least about 60% within about 24 hours, preferably having a maximum rate of release from about 10% to about 50% per hour, more preferably from about 10% to about 25% per hour. In another embodiment, the release rate in-vitro is preferably less than about 10% within about 12 hours and at least about 60% within about 24 hours, preferably having a maximum rate of release from about 10% to about 50% per hour, more preferably from about 10% to about 25% per hour. The dosage form provides a duration of therapeutic effect of about 24 hours.

Further provided for by the invention is a sustained-release oral dosage form comprising: a first subunit and a second subunit. The first subunit includes a first opioid analgesic and the second subunit includes a second opioid analgesic, wherein the first and second opioid analgesics can be the same or different. The first subunit releases substantially all of the first opioid analgesic within about 12 hours and the second subunit releases less than about 10% of the second opioid analgesic within about 6 hours and at least about 60% of the second opioid analgesic within about 24 hours. The dissolution rate in-vitro is measured by standard USP Drug Release test of U.S. Pharmacopeia XXVI (2003) <724>. In a preferred embodiment, the oral dosage form releases from about 35% to about 65% of the first and second opioid analgesic after about 10 hours. In another embodiment, the oral dosage form releases less than about 10% of the first and second opioid analgesic after about 1 hour. In yet another embodiment, the oral dosage form releases greater than about 70% of the first and second opioid analgesic after about 20 hours. The dosage form provides a duration of therapeutic effect of about 24 hours.

The invention also provides an oral dosage form comprising an opioid analgesic or salt thereof in sustained-release form, which, at steady-state, provides an in-vivo plasma profile of a maximum opioid plasma concentration (Cₘₐₓ) and an opioid plasma concentration at about 24 hours after administration (C₂₄), wherein the ratio of Cₘₐₓ to C₂₄ is less than about 2:1; a maximum opioid plasma concentration (Cₘₐₓ), and an opioid plasma concentration at about 12 hours after administration (Cₘₐₓ), and an opioid plasma concentration at about 24 hours after administration (C₂₄), wherein the average opioid plasma concentration between Cₘₐₓ and C₁₂ is substantially equal to the average opioid plasma concentration between C₁₂ and C₂₄; a first maximum opioid plasma concentration (Cₘₐₓ₁) between 0 hours and about 12 hours after administration, and a second maximum opioid plasma concentration (Cₘₐₓ₂) between 12 hours and about 24 hours after administration; a first maximum opioid plasma concentration (Cₘₐₓ₁) between 0 hours and about 12 hours after administration, a second maximum opioid plasma concentration (Cₘₐₓ₂) between 12 hours and about 24 hours after administration, and an opioid plasma concentration at about 24 hours after administration (C₂₄), wherein the average plasma opioid concentration between about Cₘₐₓ₁ and about Cₘₐₓ₂ is substantially equal to the average opioid plasma concentration between about Cₘₐₓ₂ and about C₂₄; a first opioid maximum plasma concentration and a first minimum opioid plasma concentration (Cₘᵢₙ₁) between about 0 hours and about 12 hours after administration, a second maximum opioid plasma concentration (Cₘₐₓ₂), and an opioid plasma concentration at about 24 hours after administration (C₂₄), wherein the ratio of Cₘₐₓ₁ to Cₘᵢₙ₁ is less than about 2:1 or the ratio of Cₘₐₓ₂ to C₂₄ is less than about 2:1; or a first maximum opioid plasma concentration (Cₘₐₓ₁) and a first minimum opioid plasma concentration (Cₘᵢₙ₁) between about 0 hours and about 12 hours after administration, a second opioid maximum plasma concentration (Cₘₐₓ₂), and an opioid plasma concentration at about 24 hours after administration (C₂₄), wherein the difference between the ratio of Cₘₐₓ₁ to Cₘᵢₙ₁ and the ratio of Cₘₐₓ₂ to C₂₄ is less than about 30%.

The invention provides a sustained-release oral dosage form comprising a first subunit and a second subunit, wherein the first subunit comprises a first opioid analgesic and the second subunit comprises a second opioid analgesic, wherein the first and second opioid analgesics can be the same or different; or the first subunit comprises a first opioid analgesic and a first release-retarding material and the second subunit comprises a second opioid analgesic and a second release-retarding material, wherein the first and second opioid analgesics can be the same or different, wherein the first and second release-retarding material can be the same or different. In a preferred embodiment, the opioid analgesics are the same, and the first and second release-retarding material can be the same or different, and the dosage form, at steady-state, provides a ratio of Cₘₐₓ to C₂₄ that is less than about 2:1.

Further provided by the invention is an oral dosage form comprising an opioid analgesic or salt thereof in sustained-release form, which at steady-state, provides a first Area Under the Curve (AUC₁) between 0 and about 12 hours and a second Area Under the Curve (AUC₂) between 12 hours and about 24 hours, wherein the difference between AUC₂ and AUC₁ is less than about 50%.

Still further provided by the invention is a method of treating pain. The method comprises orally administering to a human on a once-daily basis an oral sustained-release dosage form as described above.

### BRIEF DESCRIPTION OF THE DRAWINGS

The following figures are illustrative of embodiments of the invention and are not meant to limit the scope of the invention.

FIG. 1 is a graph of percent vs. time (minutes), which shows the dissolution profile of a sustained-release oral dosage form having the formulation as set forth in Table 1.

FIG. 2 is a graph of percent vs. time (minutes), which shows the dissolution profile of a sustained-release oral dosage form having the formulation as set forth in Table 2.

FIG. 3 is a graph of percent vs. time (minutes), which shows the dissolution profile of a sustained-release oral dosage form having the formulation as set forth in Table 3.

FIG. 4 is a graph of percent vs. time (minutes), which shows the dissolution profile of a sustained-release oral dosage form having the formulation as set forth in Table 4.

FIG. 5 is a graph of percent vs. time (minutes), which shows the dissolution profile of a sustained-release oral dosage form having the formulation as set forth in Table 5.

### DETAILED DESCRIPTION OF THE INVENTION

Oral dosage forms with 0 to 12 hours release profiles are known in the art, as are oral dosage forms with 12-24 hour release profiles. The invention combines two different subunits with different release profiles to achieve various functional release profiles, wherein the combination of the different subunits results in novel sustained-release oral dosage forms. The dosage forms of the invention can provide substantially constant or therapeutically consistent levels of the opioid agonist without significant increases in the intensity or degree of side effects, such as nausea, vomiting, or drowsiness, which are often associated with high blood levels of opioids. In preferred embodiments, the dosage form is efficacious in a human in the fed or fast state.

By “oral dosage form” is meant a unit dosage form prescribed or intended for oral administration.

By “sustained release” is meant to include the release of the drug (e.g., an opioid analgesic) at such a rate that blood (e.g., plasma) levels are maintained within a therapeutic range but below toxic levels for at least about 12 hours after administration at steady-state. The term “steady-state” means that a plasma level for a given drug has been achieved and is maintained with subsequent doses of the drug at a level at or above the minimum effective therapeutic level and below the minimum toxic plasma level for a given drug. For opioid analgesics, the minimum effective therapeutic level will be partially determined by the amount of pain relief achieved in a given patient. It is understood by those ordinarily skilled in the art that symptoms of pain will vary between individuals and that the measurement of signs of pain is subjective.

By “Cₘₐₓ” is meant the measured concentration of the opioid in the plasma at the point of maximum concentration.

By “C₂₄” is meant the measured concentration of the opioid in the plasma at about 24 hours.

By “C₁₂” is meant the measured concentration of the opioid in the plasma at about 12 hours.

By “Cₘₐₓ₁” is meant the measured concentration of the opioid in the plasma at the maximum point of concentration between 0 and about 12 hours.

By “Cₘₐₓ₂” is meant the measured concentration of the opioid in the plasma at the maximum point of concentration between about 12 and about 24 hours.

By “Cₘᵢₙ₁” is meant the measured concentration of the opioid in the plasma at the point of minimum concentration between 0 and about 12 hours.

By “subunit” is meant to include a composition, mixture, particle, etc., that can provide an oral dosage form when combined with other subunits, e.g., at least one additional subunit.

By “AUC” is meant the area under the curve measured from one time to another.

By “AUC₁” is meant the area under the curve measured between 0 and about 12 hours.

By “AUC₂” is meant the area under the curve measured between about 12 and about 24 hours.

“Opioid” includes a drug, hormone, or other chemical or biological substance, natural or synthetic, having a sedative, narcotic, or otherwise similar effect(s) to opium or its natural or synthetic derivatives.

By “opioid analgesic,” sometimes used herein interchangeably with terms “opioid” and “opioid analgesic,” is meant to include one or more opioid analgesics, either alone or in combination, and is further meant the base of the opioid, mixed, or combined agonist-antagonists, partial agonists, pharmaceutically acceptable salts thereof, stereoisomers thereof, ethers thereof, esters thereof, and combinations thereof. By “opioid antagonist” is meant one or more opioid antagonists, either alone or in combination, and is further meant partial antagonists, pharmaceutically acceptable salts thereof, stereoisomers thereof, ethers thereof, esters thereof, and combinations thereof.

Any suitable, pharmaceutically acceptable, opioid analgesic can be used in the present inventive oral dosage forms. Preferably, the opioid analgesic is selected from the group consisting of alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydroetorphine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, etorphine, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papavereturn, pentazocine, phenadoxone, phenazocine, phenomorphan, phenoperidine, piminodine, piritramide, propheptazine, promedol, properidine, propiram, propoxyphene, sufentanil, tramadol, tilidine, derivatives or complexes thereof, salts thereof and combinations thereof. More preferably, the opioid analgesic is selected from the group consisting of hydrocodone, hydromorphone, oxycodone, dihydrocodeine, codeine, dihydromorphine, morphine, buprenorphine, derivatives or complexes thereof, pharmaceutically acceptable salts thereof and combinations thereof. Most preferably, the opioid analgesic is morphine, oxycodone or hydrocodone.

Pharmaceutically acceptable salts of opioid analgesics include, but are not limited to, metal salts, such as sodium salt, potassium salt, cesium salt and the like; alkaline earth metals, such as calcium salt, magnesium salt and the like; organic amine salts, such as triethylamine salt, pyridine salt, picoline salt, ethanolamine salt, triethanolamine salt, dicyclohexylamine salt, N,N′-dibenzylethylenediamine salt and the like; inorganic acid salts, such as hydrochloride, hydrobromide, sulfate, phosphate and the like; organic acid salts, such as formate, acetate, trifluoroacetate, maleate, tartrate and the like; sulfonates, such as methanesulfonate, benzenesulfonate, p-toluenesulfonate, and the like; and amino acid salts, such as arginate, asparginate, glutamate and the like.

Equianalgesic doses of various opioids, in comparison to a 15 mg dose of hydrocodone, are set forth in Chart 1 below:

CHART 1
Equianalgesic Doses of Opioids

| Opioid | Calculated Dose (mg) |
|---|---|
| Oxycodone | 13.5 |
| Codeine | 90.0 |
| Hydrocodone | 15.0 |
| Hydromorphone | 3.375 |
| Levorphanol | 1.8 |
| Meperidine | 135.0 |
| Methadone | 9.0 |
| Morphine | 27.0 |

Hydrocodone is a semisynthetic narcotic analgesic and antitussive with multiple nervous system and gastrointestinal actions. Chemically, hydrocodone is 4,5-epoxy-3-methoxy-17-methylmorphinan-6-one, and is also known as dihydrocodeinone. Like other opioids, hydrocodone can be habit-forming and can produce drug dependence of the morphine type. Like other opium derivatives, excess doses of hydrocodone will depress respiration.

For use as an analgesic, hydrocodone bitartrate is commonly available in the United States only as a fixed combination with non-opiate drugs (e.g., ibuprofen, acetaminophen, aspirin; etc.) for relief of moderate to moderately severe pain. Oral hydrocodone is also available in Europe (e.g., Belgium, Germany, Greece, Italy, Luxembourg, Norway and Switzerland) as an antitussive agent. A parenteral formulation is also available in Germany as an antitussive agent.

A common dosage form of hydrocodone is in combination with acetaminophen and is commercially available in the United States from UCB Pharma, Inc. (Smyrna, Ga.), for example, as Lortab® in 2.5/500 mg, 5/500 mg, 7.5/500 mg and 10/500 mg hydrocodone/acetaminophen tablets. Tablets are also available in the ratio of 7.5 mg hydrocodone bitartrate and 650 mg acetaminophen and in the ratio of 7.5 mg hydrocodone bitartrate and 750 mg acetaminophen. Hydrocodone, in combination with aspirin, is given in an oral dosage form to adults generally in 1-2 tablets every 4-6 hours as needed to alleviate pain. The tablet form is 5 mg hydrocodone bitartrate and 224 mg aspirin with 32 mg caffeine; or 5 mg hydrocodone bitartrate and 500 mg aspirin. Another formulation comprises hydrocodone bitartrate and ibuprofen. Vicoprofen®, commercially available in the U.S. from Knoll Laboratories (Mount Olive, N.J.), is a tablet containing 7.5 mg hydrocodone bitartrate and 200 mg ibuprofen. Such formulations can be incorporated into the oral dosage forms of the invention.

Oxycodone, chemically known as 4,5-epoxy-14-hydroxy-3-methoxy-17-methylmorphinan-6-one, is an opioid analgesic whose principal therapeutic action is analgesia. Other therapeutic effects of oxycodone include anxiolysis, euphoria and feelings of relaxation. The precise mechanism of its analgesic action is not known, but specific CNS opioid receptors for endogenous compounds with opioid-like activity have been identified throughout the brain and spinal cord and play a role in the analgesic effects of this drug.

Oxycodone is commercially available in the United States, e.g., as Oxycotin® from Purdue Pharma L.P. (Stamford, Conn.), as controlled-release tablets for oral administration containing 10 mg, 20 mg, 40 mg or 80 mg oxycodone hydrochloride, and as OxyIR™, also from Purdue Pharma L.P., as immediate-release capsules containing 5 mg oxycodone hydrochloride.

With respect to all oral dosage forms discussed herein, in embodiments in which the opioid analgesic comprises hydrocodone, the sustained-release oral dosage form can comprise from about 8 mg to about 50 mg of hydrocodone per dosage unit. In sustained-release, oral dosage forms comprising hydromorphone, from about 2 mg to about 64 mg hydromorphone hydrochloride can be used. In another embodiment, the opioid analgesic comprises morphine, and the sustained-release, oral dosage form comprises from about 2.5 mg to about 800 mg morphine, by weight. In yet another embodiment, the opioid analgesic comprises oxycodone and the sustained-release, oral dosage form comprises from about 2.5 mg to about 800 mg oxycodone. In certain preferred embodiments, the sustained-release oral dosage form comprises from about 20 mg to about 30 mg oxycodone. Controlled-release oxycodone formulations are known in the art. The following documents describe various controlled-release oxycodone formulations, which can be incorporated into the oral dosage forms of the invention, and processes for their manufacture: U.S. Pat. Nos. 5,266,331; 5,549,912; 5,508,042; and 5,656,295, which are incorporated herein by reference. The opioid analgesic can comprise tramadol and the sustained-release, oral dosage forms can comprise from about 25 mg to 800 mg tramadol per dosage unit. The dosage form can comprise more than one opioid analgesic to provide a substantially equivalent therapeutic effect. Alternatively, the dosage form can comprise molar equivalent amounts of salts of the opioid analgesics.

In certain preferred embodiments of an oral dosage form discussed herein, wherein the opioid is morphine, the plasma concentration at steady-state is from about 20 ng/ml to about 30 ng/ml, and preferably is from about 23 ng/ml to about 28, ng/ml, based on a 100 mg, 24-hour dosing regimen. The preferred embodiment is linear- and dose-proportional, so that a doubling of the dose of morphine will correlate to about a doubling of the concentration in the plasma.

The compositions described herein provide specific dissolution profiles. “Dissolution profile” as used herein, means a plot of amount of active ingredient released as a function of time. The dissolution profile can be measured utilizing the Drug Release Test <724> which incorporates standard test USP (2002) (Test <711>). A profile is characterized by the test conditions selected. Thus the dissolution profile can be generated at a preselected shaft speed, temperature and pH of the dissolution media. A first dissolution profile can be measured at a pH level approximating that of the stomach. At least a second dissolution profile can be measured at pH levels approximating that of one point in the intestine or several pH levels approximating multiple points in the intestine. A highly acidic pH may simulate the stomach and a less acidic to basic pH can simulate the intestine. By the term “highly acidic pH” as used herein is meant a pH in the range of approximately 1 to 4. By the term “less acidic to basic pH” is meant a pH of greater than 4 up to approximately 7.5, preferably approximately 6 to 7.5. A pH of approximately 1.2 can be used to simulate the pH of the stomach. A pH of approximately 6.0 to 7.5, preferably 7.5, can be used to simulate the pH of the intestine. Accordingly in a further preferred aspect, a first dissolution profile is measured at a pH level approximating that of the stomach and a second dissolution profile is measured at a pH level approximating that of at least one point in the intestine; the first and second dissolution profiles for the sustained-release composition each being equal to or greater than the minimum dissolution required to provide substantially equivalent bioavailability to a capsule, tablet or liquid containing the at least one active ingredient in an immediate-release form.

The maximum dissolution rate can be measured by assessing the slope of the dissolution profile when the active ingredient is 50% dissolved with respect to the half-hour before and half-hour after about 50% dissolution is achieved. Measuring the percentage of active ingredient dissolved can be done by spectroscopy techniques, as well as other well know techniques in the art. In a preferred embodiment of all oral dosage forms mentioned herein, the maximum dissolution rate is from about 3% to about 50% per hour, more preferably from about 5% to about 40% per hour, even more preferably from about 7% to about 30% per hour, and most preferably from about 10% to about 25% per hour.

In a preferred embodiment of the invention, the oral dosage form includes a subunit comprising an opioid analgesic and a sustained-release material, wherein the dissolution rate in-vitro of the subunit is less than about 10%, preferably less than about 5%, more preferably less than about 3%, most preferably less than about 1%, within about 6 hours, preferably within about 8 hours, more preferably within about 10 hours, and most preferably within about 12 hours, and at least about 60%, preferably about 70%, more preferably about 80%, and most preferably greater than about 90%, within about 24 hours. In preferred embodiments, the maximum rate of release from about 3% to about 50% per hour, more preferably from about 5% to about 40% per hour, even more preferably from about 7% to about 30% per hour, and most preferably from about 10% to about 25% per hour.

In another embodiment, the oral dosage form includes a first subunit and a second subunit. The first subunit includes a first opioid analgesic and the second subunit includes a second opioid analgesic. The first and second opioid analgesics can be the same or different. The first subunit releases substantially all of the first opioid analgesic within about 12 hours and the second subunit releases less than about 10%, preferably less than about 5%, more preferably less than about 3%, most preferably less than about 1%, of the second opioid analgesic within about 6 hours, preferably within about 8 hours, more preferably within about 10 hours, most preferably within about 12 hours, and at least about 60%, more preferably at least about 70%, even more preferably at least about 80%, and most preferably at least about 90%, of the second opioid analgesic within about 24 hours. The dissolution rate in-vitro is measured by standard Drug Release test of U.S. Pharmacopeia XXVI (2003) <724>.

In certain preferred embodiments, the oral dosage form releases from about 30% to about 70%, preferably from about 40% to about 60%, more preferably from about 45% to about 55%, and most preferably about 50%, of the first and second opioid analgesic after about 6 hours, preferably after about 8 hours, more preferably after about 10 hours, and most preferably after about 12 hours.

In another embodiment, the oral dosage form releases less than about 10%, preferably less than about 7%, more preferably less than about 5%, most preferably less than about 3%, of the first and second opioid analgesic within about 3 hours, preferably within about 2 hours, most preferably within about 1 hour.

In yet another embodiment, the oral dosage form releases greater than about 70%, preferably greater than about 80%, more preferably greater than about 90%, most preferably greater than about 99%, of the first and second opioid analgesic after about 18, preferably after about 20 hours, more preferably after about 22 hours, and most preferably after about 24 hours.

Desirably, the dosage forms, in use, exhibit less fluctuations in plasma concentrations in active ingredient at steady-state over a 24 hour period, relative to the active ingredient in any known 24-hour formulation or in any uncoated form. Plasma opioid concentrations can be determined by a high-performance liquid chromatographic procedure or other procedures known in the art.

In a preferred embodiment, the oral dosage form provides, at steady state, a ratio of Cₘₐₓ to C₂₄ that is less than about 2:1; preferably less that about 1.9:1 (e.g., less than about 1.8:1, less than about 1.7:1, etc.), more preferably less than about 1.6:1 (e.g., less than about 1.5:1, less than about 1.4:1, etc.), and most preferably less that about 1.3:1.

In yet another embodiment, the oral dosage form provides, at steady state, an average opioid plasma concentration between Cₘₐₓ and C₁₂ that is substantially equal to the average opioid plasma concentration between C₁₂ and C₂₄.

In still a further embodiment, the oral dosage form provides, at steady state, a ratio of Cₘₐₓ₁ to Cₘᵢₙ₁ that is less than about 2:1; preferably less that about 1.9:1 (e.g., less than about 1.8:1, less than about 1.7:1, etc.), more preferably less than about 1.6:1 (e.g., less than about 1.5:1, less than about 1.4:1, etc.), and most preferably less that about 1.3:1.

In another embodiment, the oral dosage form provides, at steady state, a ratio of Cₘₐₓ₂ to C₂₄ that is less than about 2:1; preferably less that about 1.9:1 (e.g., less than about 1.8:1, less than about 1.7:1, etc.), more preferably less than about 1.6:1 (e.g., less than about 1.5:1, less than about 1.4:1, etc.), and most preferably less that about 1.3:1.

In another embodiment, the oral dosage form provides, at steady state, a difference between the ratio of Cₘₐₓ₁ to Cₘᵢₙ₁ and the ratio of Cₘₐₓ₂ to C₂₄ is less than about 50%, preferably less than about 40%, more preferably less than about 35%, and most preferably less than about 30%.

In yet another embodiment, the oral dosage form provides, at steady state, a difference between AUC₂ and AUC₁ that is less than about 70%, preferably less than about 60%, more preferably less than about 55%, and most preferably less than about 50%.

In other embodiments, the oral dosage form provides a combination of the dissolution profiles and plasma concentrations discussed herein. For example, in a preferred embodiment, the invention provides a sustained-release oral dosage form comprising a subunit, wherein the subunit comprises an opioid analgesic and a sustained release material, wherein the dissolution rate in-vitro of the subunit, when measured by standard USP Drug Release test of U.S. Pharmacopeia (2003) <724>, is less than about 10% within about 6 hours and at least about 60% within about 24 hours; less than about 10% within about 8 hours and at least about 60% within about 24 hours; less than about 10% within about 10 hours and at least about 60% within about 24 hours; or less than about 10% within about 12 hours and at least about 60% within about 24 hours; which, at steady-state, provides a maximum opioid plasma concentration (Cₘₐₓ) and an opioid plasma concentration at about 24 hours after administration (C₂₄), wherein the ratio of Cₘₐₓ to C₂₄ is less than about 2:1; a maximum opioid plasma concentration (Cₘₐₓ), and an opioid plasma concentration at about 12 hours after administration (C₁₂), and an opioid plasma concentration at about 24 hours after administration (C₂₄), wherein the average opioid plasma concentration between Cₘₐₓ and C₁₂ is substantially equal to the average opioid plasma concentration between C₁₂ and C₂₄; a first maximum opioid plasma concentration (Cₘₐₓ₁) between about 0 hours and about 12 hours after administration, and a second maximum opioid plasma concentration (C₁₂) between about 12 hours and about 24 hours after administration; a first maximum opioid plasma concentration (Cₘₐₓ₁) between about 0 hours and about 12 hours after administration, a second maximum opioid plasma concentration (Cₘₐₓ₂) between about 12 hours and about 24 hours after administration, and an opioid plasma concentration at about 24 hours after administration (C₂₄), wherein the average plasma opioid concentration between about Cₘₐₓ₁ and about Cₘₐₓ₂ is substantially equal to the average opioid plasma concentration between about Cₘₐₓ₂ and about C₂₄; a first opioid maximum plasma concentration (Cₘₐₓ₁) and a first minimum opioid plasma concentration (Cₘᵢₙ₁) between about 0 hours and about 12 hours after administration, a second maximum opioid plasma concentration (Cₘₐₓ₂), and an opioid plasma concentration at about 24 hours after administration (C₂₄), wherein the ratio of Cₘₐₓ₁ to Cₘᵢₙ₁ is less than about 2:1 or the ratio of Cₘₐₓ₂ to C₂₄ is less than about 2:1; or a first maximum opioid plasma concentration (Cₘₐₓ₁) and a first minimum opioid plasma concentration (Cₘᵢₙ₁) between about 0 hours and about 12 hours after administration, a second opioid maximum plasma concentration (Cₘₐₓ₂), and an opioid plasma concentration at about 24 hours after administration (C₂₄), wherein the difference between the ratio of Cₘₐₓ₁ to Cₘᵢₙ₁ and the ratio of Cₘₐₓ₂ to C₂₄ is less than about 30%, the dosage form providing a duration of therapeutic effect of about 24 hours.

In certain embodiments, the first subunit comprises a first opioid analgesic and a first release-retarding material and the second subunit comprises a second opioid analgesic and a second release-retarding material, wherein the first and second opioid analgesics are the same or different, and wherein the first and second release-retarding material are the same or different.

The opioid analgesic in sustained-release form is preferably a particle of opioid analgesic that is combined with a release-retarding material. The release-retarding material is desirably a material that permits release of the opioid analgesic at a sustained rate in an aqueous medium. The release-retarding material can be selectively chosen so as to achieve, in combination with the other stated properties, a desired in-vivo release rate.

Preferably, an oral dosage form of the invention is formulated to provide for an increased duration of analgesic action, allowing once-daily dosing. In general, a release-retarding material is used to provide the increased duration of analgesic action. However, it should be appreciated that the dosage form of the invention can be provided for more frequent dosage regiments, e.g., twice-daily, thrice-daily; etc.

Preferred release-retarding materials include, but are not limited to, acrylic polymers, celluloses, alkylcelluloses, shellac, zein, hydrogenated vegetable oil, hydrogenated castor oil, and combinations thereof.

In certain preferred embodiments of the oral dosage forms discussed herein, the release-retarding material is a pharmaceutically acceptable acrylic polymer, including acrylic acid and methacrylic acid copolymers, methyl methacrylate copolymers, ethoxyethyl methacrylates, cynaoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer and glycidyl methacrylate copolymers. In certain preferred embodiments, the acrylic polymer comprises one or more ammonio methacrylate copolymers. Ammonio methacrylate copolymers are well-known in the art, and are described in National Formulary XXI (“NF XXI”) as fully polymerized copolymers of acrylic and methacrylic acid esters with a low content of quaternary ammonium groups.

In other preferred embodiments, the release-retarding material is an alkyl cellulosic material, such as ethylcellulose. Those ordinarily skilled in the art will appreciate that other cellulosic polymers, including other alkyl cellulosic polymers, can be substituted for part or all of the ethylcellulose.

The release-retarding material can also include an erosion-promoting agent. Any suitable erosion promoting agent can be used. Examples of such erosion promoting agents include, but are not limited to, starches and gums; an agent that makes microporous lamina in the environment of use, such as polycarbonates comprised of linear polyesters of carbonic acid in which carbonate groups reoccur in the polymer chain; and/or a semi-permeable polymer.

The release-retarding material can also include an exit means comprising at least one passageway, orifice, or the like. The passageway can be formed by such methods as those disclosed in U.S. Pat. Nos. 3,845,770; 3,916,889; 4,063,064; and 4,088,864, which are incorporated herein by reference. The passageway can have any shape, such as round, triangular, square, elliptical, irregular, etc.

Release-modifying agents, which affect the release properties of the release-retarding material, also can be used in an oral dosage form of the invention. Any suitable release-modifying agent can be used. In a preferred embodiment, the release-modifying agent can also act to retard the release of the active. Preferably, the agent is an anionic alkyl salt, such as sodium lauryl sulfate, metal stearate, and combinations thereof. In yet another preferred embodiment, the release-modifying agent functions as a pore-former. The pore former can be organic or inorganic, and includes materials that can be dissolved, extracted or leached from the coating in the environment of use. The pore-former can comprise one or more hydrophilic polymers, such as hydroxypropylmethylcellulose, hydroxypropylcellulose, polyvinyl pyrrolidone, lactose, and combinations thereof. In particularly preferred embodiments, the release-modifying agent is hydroxypropylmethylcellulose, hydroxypropylcellulose, lactose, polyvinyl pyrrolidone, sodium lauryl sulfate, metal stearates or combinations thereof.

In certain embodiments, the opioid analgesic in sustained-release form can include a plurality of substrates comprising the active ingredient, which substrates arc coated with a sustained-release coating comprising a release-retarding material. In certain other embodiments, the opioid analgesic in sustained-release form can include a plurality of substrates comprising the analgesic in a sustained-release matrix. Combinations of the foregoing substrates, matrices and coatings are also contemplated to be within the scope of the invention provided herein.

The sustained-release preparations of the invention can be made in conjunction with any multiparticulate system, such as beads, ion-exchange resin beads, spheroids, microspheres, seeds, pellets, granules, and other multiparticulate systems in order to obtain a desired sustained-release of the opioid analgesic. The multiparticulate system can be presented in a capsule or in any other suitable unit dosage form. Further, the sustained-release preparations can be made tamper-resistant, such as those formulations disclosed in Boehm, entitled “Tamper-Resistant Oral Opioid Agonist Formulations,” filed Sep. 22, 2003, and incorporated herein by reference. In certain preferred embodiments, more than one multiparticulate system can be used, each exhibiting different characteristics, such as pH-dependence of release, time for release in various media (e.g., acid, base, simulated intestinal fluid), release in-vivo, size and composition.

In order to obtain a sustained-release of the opioid analgesic in a manner sufficient to provide an analgesic effect for the sustained durations, the substrate comprising the opioid analgesic can be coated with an amount of release-retarding material sufficient to obtain a weight gain level from about 2 to about 30%, although the coat can be greater or lesser depending upon the physical properties of the particular opioid analgesic utilized and the desired release rate, among other things. Moreover, there can be more than one release-retarding material used in the coat, as well as various other pharmaceutical excipients. Solvents typically used for coating a release-retarding material onto a substrate, for example, include pharmaceutically acceptable solvents, such as water, methanol, ethanol, methylene chloride and combinations thereof. A sustained-release oral dosage form as described herein can further comprise at least one release-modifying agent.

In certain embodiments of the oral dosage forms described herein, the release-retarding material is in the form of a coating comprising an aqueous dispersion of a hydrophobic polymer. The inclusion of an effective amount of a plasticizer in the aqueous dispersion of hydrophobic polymer will further improve the physical properties of the film. For example, because ethylcellulose has a relatively high glass transition temperature and does not form flexible films under normal coating conditions, it is necessary to plasticize the ethylcellulose before using it as a coating material. Generally, the amount of plasticizer included in a coating solution is based on the concentration of the film-former, e.g., most often from about 1 to about 50 percent by weight of the film-former. Concentrations of the plasticizer, however, can be determined by routine experimentation.

Examples of plasticizers for ethylcellulose and other celluloses include, but are not limited to, dibutyl sebacate, diethyl phthalate, dibutyl phthalate, triethyl citrate, tributyl citrate, and triacetin, although it is possible that other plasticizers (such as acetylated monoglycerides, phthalate esters, castor oil, etc.) can be used.

Examples of plasticizers for the acrylic polymers include, but are not limited to, citric acid esters such as triethyl citrate, tributyl citrate, dibutyl phthalate, and possibly polyethylene glycols, propylene glycol, diethyl phthalate, castor oil, and triacetin, although it is possible that other plasticizers (such as acetylated monoglycerides, phthalate esters, castor oil, etc.) can be used.

The sustained-release profile of drug release in the formulations of the invention (either in-vivo or in-vitro) can be altered, for example, by using more than one release-retarding material, varying the thickness of the release-retarding material, changing the particular release-retarding material used, altering the relative amounts of release-retarding material, altering the manner in which the plasticizer is added (e.g., when the sustained-release coating is derived from an aqueous dispersion of hydrophobic polymer), by varying the amount of plasticizer relative to retardant material, by the inclusion of additional ingredients or excipients, by altering the method of manufacture; etc.

In further embodiments, more than one opioid analgesic is included and/or a non-opioid drug is included. Such non-opioid drugs preferably provide analgesia, and include, for example, aspirin, acetaminophen, non-steroidal anti-inflammatory drugs (“NSAIDs”), N-methyl-D-aspartate (“NMDA”) receptor antagonists, cyclooxygenase-II inhibitors (“COX-II inhibitors”), and glycine receptor antagonists.

Exemplary NSAIDs include ibuprofen, diclofenac, naproxen, benoxaprofen, flurbiprofen, fenoprofen, flubufen, ketoprofen, indoprofen, piroprofen, carprofen, oxaprozin, pramoprofen, muroprofen, trioxaprofen, suprofen, aminoprofen, tiaprofenic acid, fluprofen, bucloxic acid, indomethacin, sulindac, tolmetin, zomepirac, tiopinac, zidometacin, acemetacin, fentiazac, clidanac, oxpinac, mefenamic acid, meclofenamic acid, flufenamic acid, niflumic acid, tolfenamic acid, diflurisal, flufenisal, piroxicam, sudoxicam or isoxicam, and the like. Useful dosages of these drugs are well known.

Exemplary NMDA receptor antagonists include morphinans, such as dextromethorphan or dextrophan, ketamine, d-methadone, and pharmaceutically acceptable salts thereof, and encompasses drugs that block a major intracellular consequence of NMDA-receptor activation, e.g., a ganglioside such as (6-aminothexyl)-5-chloro-1-naphthalenesulfonamide. These drugs are stated to inhibit the development of tolerance to and/or dependence on addictive drugs, e.g., narcotic analgesics such as morphine, codeine, etc., in U.S. Pat. Nos. 5,321,012 and 5,556,838 (both to Mayer et al.), and to treat chronic pain in U.S. Pat. No. 5,502,058 (Mayer et al.), and are incorporated herein by reference. The NMDA agonist can be included alone or in combination with a local anesthetic such as lidocaine, as described in these Mayer et al. patents.

COX-II inhibitors have been reported in the art and many chemical compounds are known to produce inhibition of cyclooxygenase-II. COX-II inhibitors are described, for example, in U.S. Pat. Nos. 5,616,601; 5,604,260; 5,593,994; 5,550,142; 5,536,752; 5,521,213; 5,475,995; 5,639,780; 5,604,253; 5,552,422, 5,510,368; 5,436,265; 5,409,944 and 5,130,311, and are incorporated herein by reference. Certain preferred COX-II inhibitors include celecoxib (SC-58635), DUP-697, flosulide (CGP-28238), meloxicam, 6-methoxy-2-naphthylacetic acid (6-NMA), MK-966 (also known as Vioxx), nabumetone (prodrug for 6-MNA), nimesulide, NS-398, SC-5766, SC-58215, T-614, or combinations thereof. Dosage levels of COX-II inhibitor on the order of from about 0.005 mg to about 140 mg per kilogram of body weight per day has been shown to be therapeutically effective in combination with an opioid analgesic. Alternatively, about 0.25 mg to about 7 g per patient per day of a COX-II inhibitor can be administered in combination with an opioid analgesic.

The treatment of chronic pain via the use of glycine receptor antagonists and the identification of such drugs is described in U.S. Pat. No. 5,514,680 (Weber et al.), which is incorporated herein by reference.

In yet further embodiments, a non-opioid drug can be included which provides a desired effect other than analgesia, e.g., antitussive, expectorant, decongestant, antihistamine drugs, and the like.

In certain other embodiments, the oral dosage form can utilize a multiparticulate sustained-release matrix.

In certain embodiments, the sustained-release matrix comprises a hydrophilic and/or hydrophobic polymer, such as gums, cellulose ethers, acrylic resins and protein-derived materials. Of these polymers, the cellulose ethers, specifically hydroxyalkylcelluloses and carboxyalkylcelluloses, are preferred. The oral dosage form can contain between about 1% and about 80% (by weight) of at least one hydrophilic or hydrophobic polymer.

The hydrophobic material is preferably selected from the group consisting of alkylcellulose, acrylic and methacrylic acid polymers and copolymers, shellac, zein, hydrogenated castor oil, hydrogenated vegetable oil, or mixtures thereof. Preferably, the hydrophobic material is a pharmaceutically acceptable acrylic polymer, including acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer, poly(methyl methacrylate), poly(methacrylic acid) (anhydride), polymethacrylate, polyacrylamide, poly(methacrylic acid anhydride), and glycidyl methacrylate copolymers. In other embodiments, the hydrophobic material can also include hydroxyalkylcelluloses, such as hydroxypropylmethylcellulose, and mixtures of the foregoing.

Preferred hydrophobic materials are water-insoluble with more or less pronounced hydrophilic and/or hydrophobic trends. Preferably, the hydrophobic material has a melting point from about 30° C. to about 200° C., more preferably from about 45° C. to about 90° C. The hydrophobic material can include neutral or synthetic waxes, fatty alcohols (such as lauryl, myristyl, stearyl, cetyl or, preferably, cetostearyl alcohol), fatty acids, including fatty acid esters, fatty acid glycerides (mono-, di-, and tri-glycerides), hydrogenated fats, hydrocarbons, normal waxes, stearic acid, stearyl alcohol and hydrophobic and hydrophilic materials having hydrocarbon backbones. Suitable waxes include beeswax, glycowax, castor wax, carnauba wax and wax-like substances, e.g., a material normally solid at room temperature and having a melting point of from about 30° C. to about 100° C.

Preferably, a combination of two or more hydrophobic materials is included in the matrix formulations. If an additional hydrophobic material is included, it is preferably a natural or synthetic wax, a fatty acid, a fatty alcohol, or a mixture thereof. Examples include beeswax, carnauba wax, stearic acid and stearyl alcohol.

In certain embodiments, the sustained-release matrix comprises digestible, long chain (e.g., C₈-C₅₀, preferably C₁₂-C₄₀), substituted or unsubstituted hydrocarbons, such as fatty acids, fatty alcohols, glyceryl esters of fatty acids, mineral and vegetable oils and waxes. Hydrocarbons having a melting point of between about 25° C. and about 90° C. are preferred. Of these long chain hydrocarbon materials, fatty (aliphatic) alcohols are preferred. The oral dosage form can contain up to about 60% (by weight) of at least one digestible, long-chain hydrocarbon. Further, the sustained-release matrix can contain up to 60% (by weight) of at least one polyalkylene glycol.

In a preferred embodiment, the matrix comprises at least one water-soluble hydroxyalkyl cellulose, at least one C₁₂-C₃₆, preferably C₁₄-C₂₂, aliphatic alcohol and, optionally, at least one polyalkylene glycol. The at least one hydroxyalkyl cellulose is preferably a hydroxy (C₁-C₆) alkyl cellulose, such as hydroxypropylcellulose, hydroxypropylmethylcellulose and, preferably, hydroxyethyl cellulose. The amount of the at least one hydroxyalkyl cellulose in the oral dosage form will be determined, amongst other things, by the precise rate of opioid release required. The amount of the at least one aliphatic alcohol in the present oral dosage form will be determined by the precise rate of opioid release required. However, it will also depend on whether the at least one polyalkylene glycol is absent from the oral dosage form.

A spheronizing agent, together with the active ingredient, can be spheronized to form spheroids in certain embodiments. Microcrystalline cellulose and hydrous lactose impalpable are examples of such agents. Additionally (or alternatively), the spheroids can contain a water-insoluble polymer, preferably an acrylic polymer, an acrylic copolymer, such as a methacrylic acid-ethyl acrylate copolymer, or ethyl cellulose. In such embodiments, the sustained-release coating will generally include a water-insoluble material, such as (a) a wax, either alone or in admixture with a fatty alcohol, or (b) shellac or zein.

In addition to the above ingredients, a sustained-release matrix also can contain suitable quantities of other materials, e.g., diluents, lubricants, binders, granulating aids, colorants, flavorants and glidants that are conventional in the pharmaceutical art.

Spheroids or beads, coated with an active ingredient can be prepared, for example, by dissolving the active ingredient in water and then spraying the solution onto a substrate, for example, nu pariel 18/20 beads, using a Wurster insert. Optionally, additional ingredients are also added prior to coating the beads in order to assist the active ingredient in binding to the substrates, and/or to color the solution; etc. The resulting substrate-active material may be optionally overcoated with a barrier material to separate the therapeutically active agent from the next coat of material, e.g., release-retarding material. Preferably, the barrier material is a material comprising hydroxypropyl methylcellulose. However, any film-former known in the art can be used. Preferably, the barrier material does not affect the dissolution rate of the final product.

Pellets comprising an active ingredient can be prepared, for example, by a melt pelletization technique. Typical of such techniques is when the active ingredient in finely divided form is combined with a binder (also in particulate form) and other optional inert ingredients, and thereafter the mixture is pelletized, e.g., by mechanically working the mixture in a high shear mixer to form the pellets (e.g., pellets, granules, spheres, beads; etc., collectively referred to herein as “pellets”). Thereafter, the pellets can be sieved in order to obtain pellets of the requisite size. The binder material is preferably in particulate form and has a melting point above about 40° C. Suitable binder substances include, for example, hydrogenated castor oil, hydrogenated vegetable oil, other hydrogenated fats, fatty alcohols, fatty acid esters, fatty acid glycerides, and the like.

The diameter of the extruder aperture or exit port also can be adjusted to vary the thickness of the extruded strands. Furthermore, the exit part of the extruder need not be round; it can be oblong, rectangular; etc. The exiting strands can be reduced to particles using a hot wire cutter, guillotine; etc.

The melt-extruded multiparticulate system can be, for example, in the form of granules, spheroids, pellets, or the like, depending upon the extruder exit orifice. The terms “melt-extruded multiparticulate(s)” and “melt-extruded multiparticulate system(s)” and “melt-extruded particles” are used interchangeably herein and include a plurality of subunits, preferably within a range of similar size and/or shape. The melt-extruded multiparticulates are preferably in a range of from about 0.1 to about 12 mm in length and have a diameter of from about 0.1 to about 5 mm. In addition, the melt-extruded multiparticulates can be any geometrical shape within this size range. Alternatively, the extrudate can simply be cut into desired lengths and divided into unit doses of the therapeutically active agent without the need of a spheronization step.

The substrate also can be prepared via a granulation technique. Generally, melt-granulation techniques involve melting a normally solid hydrophobic material, e.g., a wax, and incorporating an active ingredient therein. To obtain a sustained-release dosage form, it can be necessary to incorporate an additional hydrophobic material.

A coating composition can be applied onto a substrate by spraying it onto the substrate using any suitable spray equipment. For example, a Wurster fluidized-bed system can be used in which an air jet, injected from underneath, fluidizes the coated material and effects drying, while the insoluble polymer coating is sprayed on. The thickness of the coating will depend on the characteristics of the particular coating composition, the optimum thickness for an optimum dosage being subject of routine experimentation.

The subunits, dosage forms and other components of the invention can be provided by preparing them consistently with the methods described herein or by other methods known or apparent to those skilled in the art.

In preferred embodiments, oral dosage forms are prepared to include an effective amount of melt-extruded subunits in the form of multiparticulates within a capsule. For example, a plurality of the melt-extruded multiparticulates can be placed in a gelatin capsule in an amount sufficient to provide an effective release dose when ingested and contacted by gastric fluid.

In another preferred embodiment, ingredients can be compressed into an oral tablet using conventional tableting equipment using standard techniques, Techniques and compositions for making tablets (compressed and molded), capsules (hard and soft gelatin), and pills are also described in Remington's Pharmaceutical Sciences, (Authur Osol., editor), 1553-1593 (1980), and are incorporated herein by reference. Excipients in tablet formulation can include, for example, an inert diluent such as lactose, granulating and disintegrating agents, such as cornstarch, binding agents, such as starch, and lubricating agents, such as magnesium stearate.

In yet another preferred embodiment, the therapeutically active agents are added during the extrusion process. The extrudate can be shaped into tablets as set forth in U.S. Pat. No. 4,957,681 (Klimesch et al.), which is incorporated herein by reference.

Optionally, the sustained-release, melt-extruded, multiparticulate systems or tablets can be coated, or the gelatin capsule can be further coated, with a sustained-release coating, such as the sustained-release coatings described herein. Such coatings are particularly useful when the subunit comprises bioavailable opioid analgesics, but not in sustained-release form. The coatings preferably include a sufficient amount of a hydrophobic material to obtain a weight gain level from about 2 to about 30 percent, although the overcoat can be greater, depending upon the physical properties of the particular opioid analgesic utilized and the desired release rate, among other things.

The melt-extruded dosage forms can further include combinations of melt-extruded multiparticulates containing one or more of the therapeutically active agents before being encapsulated. Furthermore, the dosage forms can also include an amount of an immediate-release opioid analgesic for prompt therapeutic effect. The immediate-release opioid analgesic can be incorporated into or coated onto the surface of the subunits after preparation of the dosage forms (e.g., controlled-release coating or matrix-based). The dosage forms can also contain a combination of controlled-release beads and matrix multiparticulates to achieve a desired effect.

The sustained-release formulations preferably slowly release the opioid analgesic, e.g., when ingested and exposed to gastric fluids, and then to intestinal fluids. The sustained release profile of the melt-extruded formulations can be altered, for example, by varying the amount of retardant, e.g., hydrophobic material, by varying the amount of plasticizer relative to hydrophobic material, by the inclusion of additional ingredients or excipients, by altering the method of manufacture; etc.

In other embodiments, the melt-extruded material is prepared without the inclusion of the therapeutically active ingredients, which are added thereafter to the extrudate. Such formulations can have the therapeutically active ingredients blended together with the extruded matrix material, and then the mixture is tableted in order to provide a slow release of the actives, e.g., the opioid analgesics. Such formulations can be particularly advantageous, for example, when the therapeutically active agent included in the formulation is sensitive to temperatures needed for softening the hydrophobic material and/or the retardant material.

The invention also provides a method comprising orally administering to a fed or unfed human on a once-daily basis an oral sustained-release dosage form of the invention, whereupon pain in the human is treated. The administration of the sustained-release dosage form is continued over the dosing interval of a unit dose to maintain an adequate pharmacodynamic response with the sustained-release dosage form. Preferably the adequate pharmacodynamic response will last between about 12 and about 24 hours, most preferably about 24 hours or greater. The administration of the sustained-release unit dosage form is continued over the dosing interval of the unit dose to maintain the adequate pharmacodynamic response with the sustained-release dosage form. If necessary, the above steps are repeated until a determination of adequate pharmacodynamic response is obtained with the sustained-release unit dosage form.

According to the above method, a patient can be titrated with a sustained-release opioid analgesic dosage form. Subsequent maintenance therapy can be provided with the same sustained-release dosage form.

In one embodiment of the invention, an oral sustained-release dosage form, as described herein, orally administered to treat pain in a human, further comprises at least one release-retarding agent. Preferably, the oral dosage form further comprises at least one release-retarding agent and at least one plasticizer, and optionally at least one release-modifying agent.

### EXAMPLES

The following examples serve to illustrate the invention and are not intended to limit its scope in any way. In the following examples, the pharmaceutical composition: includes two distinct subunits in the form of pellets (e.g., pellets, beads, spheroids, granules, etc.), a first-releasing pellet that releases opioid in a sustained manner beginning in the first 12 hours after administration to the patient and a second-releasing pellet that releases opioid in a sustained manner beginning in the second 12 hours after administration to the patient. The first-releasing pellet and the second releasing pellet can contain the same or different amounts of opioid relative to each other, can include the same or different release-retarding materials (either by type or amount), and can include the same or different excipients (either by type or amount).

In making the pellets for the first releasing-pellet and the second releasing pellet of the examples, the core element of the pharmaceutical composition includes an effective amount of at least one active ingredient and, optionally, at least one core seed, and at least one binding agent. The active ingredient is typically of high solubility.

The core can be coated with one or more of a release-retarding material. These coatings can be applied in conventional methods that will coat the core, such a spray-coating. Spray coating of core elements can be undertaken utilizing bottom, top or tangentially located spray nozzles. A bottom spray nozzle can reside proximate to the base of the fluidized bed facing upwards while a top spraying nozzle is located above the contents of the bed and facing downwards. The spray nozzle can reside in the mid-section of the fluidized bed and be oriented such as to spray tangentially to the rotating core elements.

The active ingredient is present in any suitable effective amount. The amount of active ingredient is dependent on the potency of the active ingredient and on the desired dosage strength and volume of a unit dose of the drug product. The active ingredient can be present in amounts of approximately 0.1 to 95% by weight, based on the total weight of the core element. The active ingredient is preferably a morphine compound.

A binding agent is present in amounts of from approximately 0.1 to 45% by weight, preferably approximately 0.1 to 20% by weight, more preferably approximately 3 to 15% by weight, based on the total weight of the core element. The binding agent can be of any suitable type. Suitable binders include, but are not limited to: polyvinyl pyrrolidone, hydroxypropyl cellulose, hydroxypropyl methylcellulose, methylcellulose and hydroxyethyl cellulose, sugars and mixtures thereof. The binding agent can be provided in the form of a granulating solution. An aqueous or organic solvent can be included. Methanol, ethanol or mixtures thereof may be used as solvents.

The size and amount of the core seed can vary substantially from approximately 100 um to 1700 um depending upon the amount of active ingredient to be included. Accordingly, the core seeds can vary from approximately 5 to 99% by weight, preferably 40 to 90% by weight based on the total weight of the core element, depending on the potency of the active ingredient. The core seed can be of such a diameter to provide a final core element having a diameter of approximately 200 to 2000 um.

The core seed can be of any suitable type. A sugar or an active core seed can be used. The core element can further include other carriers or excipients, fillers, stabilizing agents and colorants. Suitable fillers may be selected from insoluble materials such as silicon dioxide, talc, titanium dioxide, alumina, starch, kaolin, polacrilin potassium, powdered cellulose, and microcrystalline cellulose and mixtures thereof. Soluble fillers can be selected from mannitol, sucrose, lactose, dextrose, sodium chloride, sorbitol and mixtures thereof.

Sustained-release oral dosage forms comprising a first subunit and a second subunit were prepared as set forth in Tables 1-5.

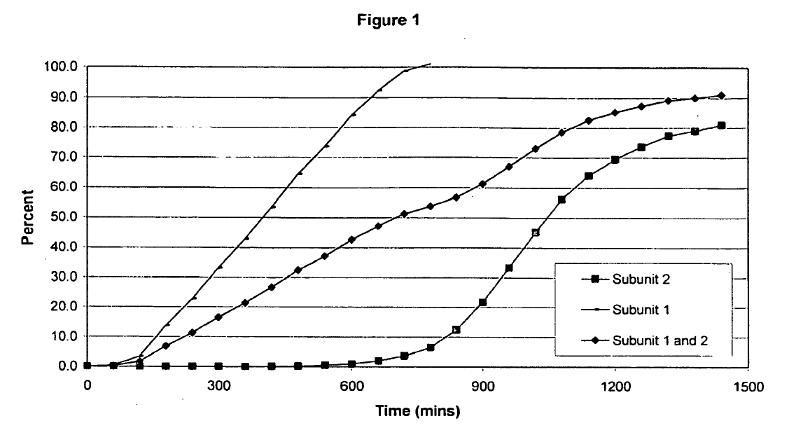

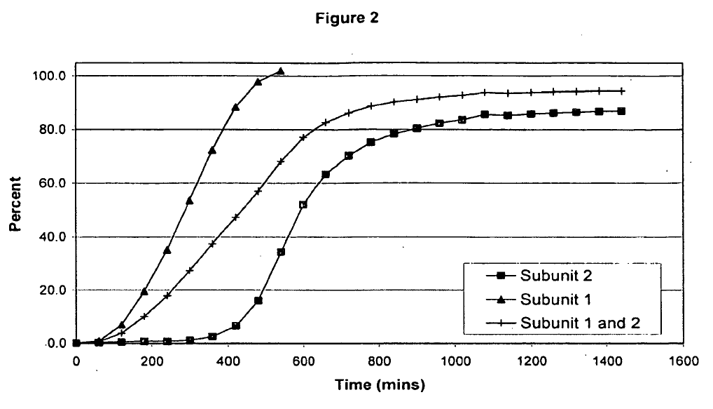

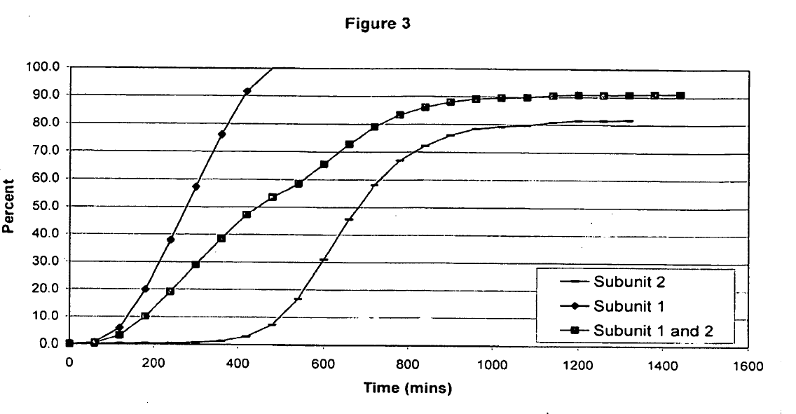

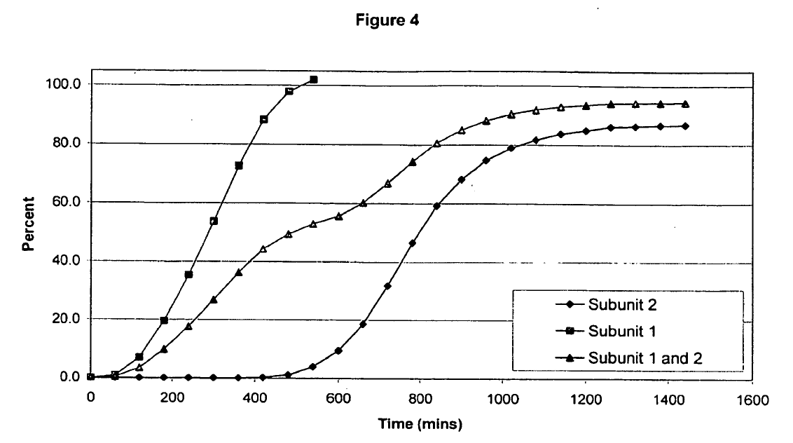

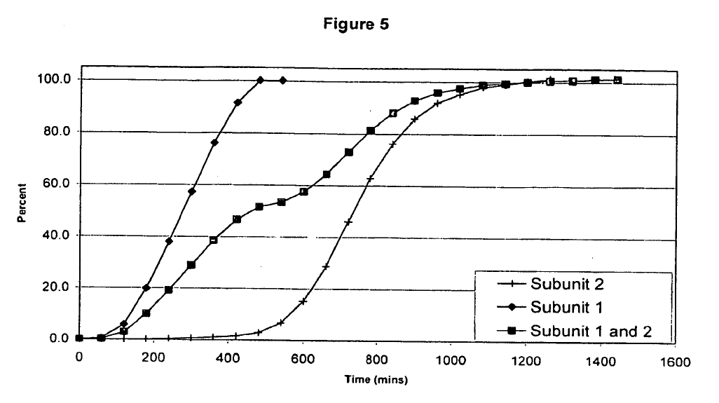

The dissolution profiles of these formulations are set forth in FIGS. 1-5, respectively. The dissolution conditions are as follows: For subunit 1, USP Basket Method (Apparatus 1) is used at 50 rpm with 500 mL of 0.1 N HCl for 1 hour followed by 500 mL of pH 7.5 0.05 M Phosphate Buffer all at 37° C. Percent release is determined by UV analysis at 286 nm with a 1 cm pathlength cell. For subunit 2, USP Paddle Method (Apparatus 2) is used at 100 rpm with 900 mL of pH 7.5 0.05 M Phosphate Buffer at 37° C. Percent release is determined by UV analysis at 286 nm with a 1 cm path length cell. Dissolution profiles for subunit 1 and 2 are obtained by the addition of equal parts of the respective dissolution profiles from subunit 1 and subunit 2.

TABLE 1

Amount per unit (mg)

First subunit:

| | |
|---|---|
| Morphine sulfate | 50.0 |
| Non-pareil seed (#16-18 mesh) | 131.9 |
| Hypromellose | 3.3 |
| Ethylcellulose | 28.1 |
| Polyethylene glycol 6000 | 9.9 |
| Eudragit L100-55 | 8.3 |
| Diethyl phthalate | 5.7 |
| Talc | 26.0 |
| Total | 263.3 |

Second subunit:

| | |
|---|---|
| Morphine sulfate | 50.0 |
| Non-pareil seed (#16-18 mesh) | 131.9 |
| Hypromellose | 3.3 |
| Eudragit RS PO | 26.9 |
| Eudragit RL PO | 4.5 |
| Triethyl citrate | 3.1 |
| Sodium lauryl sulfate | 0.7 |
| Talc | 17.6 |
| Total | 238.0 |

The first subunit was prepared by first dispersing morphine sulfate in a hydroalcoholic solution of hypromellose by mechanical stirrer and applied onto non-pareil seed by a rotor granulation process to produce morphine sulfate cores. Then a polymer solution of ethylcellulose, polyethylene glycol, Eudragit and diethyl phthalate was prepared in ethanol, and talc was dispersed uniformly into the polymer solution. The resulting polymer solution was immediately sprayed onto the morphine sulfate cores using a Wurster process, therein completing the first subunit of the oral dosage form. The second subunit was prepared by dispersing morphine sulfate in a hydroalcoholic solution of hypromellose by mechanical stirrer, and applying the resulting solution onto non-pareil seeds by rotor granulation process. Then, a polymer solution of Eudragit RS, Eudragit RL, triethyl citrate and sodium lauryl sulfate was prepared in ethanol, and talc was dispersed into the polymer solution. The resulting polymer solution was immediately sprayed onto morphine sulfate cores using a Wurster process, therein completing the second subunit of the oral dosage form.

TABLE 2

Amount per unit (mg)

First subunit:

| | |
|---|---|
| Morphine sulfate | 50.0 |
| Non-pareil seed (#16-18 mesh) | 131.9 |
| Hypromellose | 3.3 |
| Ethylcellulose | 19.9 |
| Polyethylene glycol 6000 | 6.7 |
| Eudragit L100-55 | 5.6 |
| Diethyl phthalate | 3.9 |
| Talc | 17.6 |
| Total | 238.0 |

Second subunit:

| | |
|---|---|
| Morphine sulfate | 50.0 |
| Non-pareil seed (#20-25 mesh) | 128.9 |
| Hydroxypropyl cellulose | 6.3 |
| Eudragit RS PO | 42.6 |
| Eudragit RL PO | 2.0 |
| Triethyl citrate | 4.3 |
| Sodium lauryl sulfate | 0.3 |
| Talc | 24.6 |
| Total | 259.0 |

The first subunit was prepared by first dispersing morphine sulfate in a hydroalcoholic solution of hypromellose by mechanical stirrer and applied onto non-pareil seed by a rotor granulation process to produce morphine sulfate cores. Then a polymer solution of ethylcellulose, polyethylene glycol, Eudragit and diethyl phthalate was prepared in ethanol, and talc was dispersed uniformly into the polymer solution. The resulting polymer solution was immediately sprayed onto the morphine sulfate cores using a Wurster process, therein completing the first subunit of the oral dosage form. The second subunit was prepared by dispersing morphine sulfate in a hydroalcoholic solution of hyproxypropylcellulose by mechanical stirrer, and applying the resulting solution onto non-pareil seeds by rotor granulation process. Then, a polymer solution of Eudragit RS, Eudragit RL, triethyl citrate and sodium lauryl sulfate was prepared in ethanol, and talc was dispersed into the polymer solution. The resulting polymer solution was immediately sprayed onto morphine sulfate cores using a Wurster process, therein completing the second subunit of the oral dosage form.

TABLE 3

Amount per unit (mg)

First subunit:

| | |
|---|---|
| Morphine sulfate | 50.0 |
| Non-pareil seed (#18-20 mesh) | 30.7 |
| Hypromellose | 2.7 |
| Ethylcellulose | 8.3 |
| Polyethylene glycol 6000 | 3.4 |
| Eudragit L100-55 | 2.6 |
| Diethyl phthalate | 1.8 |
| Talc | 7.9 |
| Total | 107.1 |

Second subunit:

| | |
|---|---|
| Morphine sulfate | 50.0 |
| Non-pareil seed (#20-25 mesh) | 23.4 |
| Polyvinylpyrrolidone | 10.0 |
| Eudragit RS PO | 13.9 |
| Eudragit RL PO | 1.0 |
| Triethyl citrate | 1.5 |
| Sodium lauryl sulfate | 0.3 |
| Hydroxypropyl cellulose | 1.7 |
| Talc | 9.2 |
| Total | 111.0 |

The first subunit was prepared by first dispersing morphine sulfate in a hydroalcoholic solution of hypromellose by mechanical stirrer and applied onto non-pareil seed by a rotor granulation process to produce morphine sulfate cores. Then a polymer solution of ethylcellulose, polyethylene glycol, Eudragit and diethyl phthalate was prepared in ethanol, and talc was dispersed uniformly into the polymer solution. The resulting polymer solution was immediately sprayed onto the morphine sulfate cores using a Wurster process, therein completing the first subunit of the oral dosage form. The second subunit was prepared by dispersing morphine sulfate in a hydroalcoholic solution of polyvinyl pyrrolidone by mechanical stirrer, and applying the resulting solution onto non-pareil seeds by rotor granulation process. Then, a polymer solution of Eudragit RS, Eudragit RL, triethyl citrate, sodium lauryl sulfate and hydroxypropylcellulose was prepared in ethanol, and talc was dispersed into the polymer solution. The resulting polymer solution was immediately sprayed onto morphine sulfate cores using a Wurster process, therein completing the second subunit of the oral dosage form.

TABLE 4

Amount per unit (mg)

First subunit:

| | |
|---|---|
| Morphine sulfate | 50.0 |
| Non-pareil seed (#16-18 mesh) | 131.9 |
| Hypromellose | 3.3 |
| Ethylcellulose | 19.9 |
| Polyethylene glycol 6000 | 6.7 |
| Eudragit L100-55 | 5.6 |
| Diethyl phthalate | 3.9 |
| Talc | 17.6 |
| Total | 238.0 |

Second subunit:

| | |
|---|---|
| Morphine sulfate | 50.0 |
| Non-pareil seed (#20-25 mesh) | 128.9 |
| Hypromellose | 6.3 |
| Eudragit RS PO | 54.1 |
| Eudragit RL PO | 1.9 |
| Triethyl citrate | 5.4 |
| Sodium lauryl sulfate | 0.4 |
| Talc | 30.9 |
| Total | 277.8 |

The first subunit was prepared by first dispersing morphine sulfate in a hydroalcoholic solution of hypromellose by mechanical stirrer and applied onto non-pareil seed by a rotor granulation process to produce morphine sulfate cores. Then a polymer solution of ethylcellulose, polyethylene glycol, Eudragit and diethyl phthalate was prepared in ethanol, and talc was dispersed uniformly into the polymer solution. The resulting polymer solution was immediately sprayed onto the morphine sulfate cores using a Wurster process, therein completing the first subunit of the oral dosage form. The second subunit was prepared by dispersing morphine sulfate in a hydroalcoholic solution of hypromellose by mechanical stirrer, and applying the resulting solution onto non-pareil seeds by rotor granulation process. Then, a polymer solution of Eudragit RS, Eudragit RL, triethyl citrate and sodium lauryl sulfate was prepared in ethanol, and talc was dispersed into the polymer solution. The resulting polymer solution was immediately sprayed onto morphine sulfate cores using a Wurster process, therein completing the second subunit of the oral dosage form.

TABLE 5

Amount per unit (mg)

First subunit:

| | |
|---|---|
| Morphine sulfate | 50.0 |
| Non-pareil seed (#18-20 mesh) | 30.7 |
| Hypromellose | 2.7 |
| Ethylcellulose | 8.3 |
| Polyethylene glycol 6000 | 3.4 |
| Eudragit L100-55 | 2.6 |
| Diethyl phthalate | 1.8 |
| Talc | 7.9 |
| Total | 107.1 |

Second subunit:

| | |
|---|---|
| Morphine sulfate | 50 |
| Non-pareil seed (#20-25 mesh) | 25.9 |
| Hypromellose | 2.5 |
| Fumaric acid | 5.0 |
| Eudragit RS PO | 13.9 |
| Eudragit RL PO | 1.0 |
| Triethyl citrate | 1.5 |
| Sodium lauryl sulfate | 0.3 |
| Hydroxypropyl cellulose | 1.7 |
| Talc | 9.2 |
| Total | 111.0 |

The first subunit was prepared by first dispersing morphine sulfate in a hydroalcoholic solution of hypromellose by mechanical stirrer and applied onto non-pareil seed by a rotor granulation process to produce morphine sulfate cores. Then a polymer solution of ethylcellulose, polyethylene glycol, Eudragit and diethyl phthalate was prepared in ethanol, and talc was dispersed uniformly into the polymer solution. The resulting polymer solution was immediately sprayed onto the morphine sulfate cores using a Wurster process, therein completing the first subunit of the oral dosage form. The second subunit was prepared by dispersing morphine sulfate in a hydroalcoholic solution of hypromellose and fumaric acid by mechanical stirrer, and applying the resulting solution onto non-pareil seeds by rotor granulation process. Then, a polymer solution of Eudragit RS, Eudragit RL, triethyl citrate, hydroxypropylcellulose and sodium lauryl sulfate was prepared in ethanol, and talc was dispersed into the polymer solution. The resulting polymer solution was immediately sprayed onto morphine sulfate cores using a Wurster process, therein completing the second subunit of the oral dosage form.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

1. A sustained-release oral dosage form comprising two different subunits that each comprise an opioid analgesic and a sustained-release material such that the subunits have different dissolution profiles, wherein the dissolution rate in-vitro of the oral dosage form, when measured by standard USP Drug Release test of U.S. Pharmacopeia (2003) <724>, is less than about 10% within about 6 hours and at least about 60% within about 24 hours, the dosage form providing a duration of therapeutic effect of about 24 hours.

2. The oral dosage form of claim 1, wherein the opioid analgesic is selected from the group consisting of morphine, oxycodone, hydrocodone, or any combination thereof.

3. The oral dosage form of claim 1, wherein the opioid analgesic is morphine.

4. The oral dosage form of any one of claim 1, 2, or 3, which further comprises at least one release-retarding material.

5. The oral dosage form of claim 4, wherein the release-retarding material is selected from the group consisting of acrylic polymers, cellulose, alkylcelluloses, shellac, zein, hydrogenated vegetable oil, hydrogenated castor oil, and combinations thereof.

6. The oral dosage form of claim 4, which further comprises a plasticizer.

7. The oral dosage form of claim 5, wherein the plasticizer is selected from the group consisting of dibutyl sebacate, diethyl phthalate, dibutyl phthalate, triethyl citrate, tributyl citrate, triacetin, castor oil, polyethylene glycols, and propylene glycol.

8. The oral dosage form of claim 4, which further comprises at least one release-modifying agent.

9. The oral dosage form of claim 6, which further comprises at least one release-modifying agent.

10. The oral dosage form of claim 8 or 9, wherein the release-modifying agent is selected from the group consisting of hydroxypropylmethylcellulose, lactose, hydroxypropylcellulose, polyvinyl pyrrolidone, sodium lauryl sulfate, metal stearates, and combinations thereof.

11. The oral dosage form of claim 1, wherein the maximum dissolution rate is from about 10% to about 25% per hour.

12. The oral dosage form of claim 1, wherein the maximum dissolution rate is from about 10% to about 50% per hour.

13. The oral dosage form of claim 1, wherein the dissolution rate in-vitro of the subunit is less than about 10% within about 6 hours and at least about 60% within about 24 hours and the maximum dissolution rate is from about 10% to about 25% per hour.

14. The oral dosage form of claim 1, wherein the dissolution rate in-vitro of the subunit is less than about 10% within about 6 hours and at least about 60% within about 24 hours and the maximum dissolution rate is from about 10% to about 50% per hour.

15. The oral dosage form of claim 1, wherein the dissolution rate in-vitro of the subunit is less than about 10% within about 8 hours and at least about 60% within about 24 hours and the maximum dissolution rate is from about 10% to about 25% per hour.

16. The oral dosage form of claim 1, wherein the dissolution rate in-vitro of the subunit is less than about 10% within about 8 hours and at least about 60% within about 24 hours and the maximum dissolution rate is from about 10% to about 50% per hour.

17. The oral dosage form of claim 1, wherein the dissolution rate in-vitro of the subunit is less than about 10% within about 10 hours and at least about 60% within about 24 hours and the maximum dissolution rate is from about 10% to about 25% per hour.

18. The oral dosage form of claim 1, wherein the dissolution rate in-vitro of the subunit is less than about 10% within about 10 hours and at least about 60% within about 24 hours and the maximum dissolution rate is from about 10% to about 50% per hour.

19. The oral dosage form of claim 1, wherein the dissolution rate in-vitro of the subunit is less than about 10% within about 12 hours and at least about 60% within about 24 hours and the maximum dissolution rate is from about 10% to about 25% per hour.

20. The oral dosage form of claim 1, wherein the dissolution rate in-vitro of the subunit is less than about 10% within about 12 hours and at least about 60% within about 24 hours and the maximum dissolution rate is from about 10% to about 50% per hour.

21. The oral dosage form of claim 1, which, at steady-state, provides:
a. a maximum opioid plasma concentration (Cₘₐₓ) and an opioid plasma concentration at about 24 hours after administration (C₂₄), wherein the ratio of Cₘₐₓ to C₂₄ is less than about 2:1;
b. a maximum opioid plasma concentration (Cₘₐₓ), and an opioid plasma concentration at about 12 hours after administration (C₁₂), and an opioid plasma concentration at about 24 hours after administration (C₂₄), wherein the average opioid plasma concentration between Cₘₐₓ and C₁₂ is substantially equal to the average opioid plasma concentration between C₁₂ and C₂₄;
c. a first maximum opioid plasma concentration (Cₘₐₓ₁) between about 0 hours and about 12 hours after administration, and a second maximum opioid plasma concentration (Cₘₐₓ₂) between about 12 hours and about 24 hours after administration;
d. a first maximum opioid plasma concentration (Cₘₐₓ₁) between about 0 hours and about 12 hours after administration, a second maximum opioid plasma concentration (Cₘₐₓ₂) between about 12 hours and about 24 hours after administration, and an opioid plasma concentration at about 24 hours after administration (C₂₄), wherein the average plasma opioid concentration between about Cₘₐₓ₁ and about Cₘₐₓ₂ is substantially equal to the average opioid plasma concentration between about Cₘₐₓ₂ and about C₂₄;
e. a first opioid maximum plasma concentration (Cₘₐₓ₁) and a first minimum opioid plasma concentration (Cₘᵢₙ₁) between about 0 hours and about 12 hours after administration, a second maximum opioid plasma concentration (Cₘₐₓ₂), and an opioid plasma concentration at about 24 hours after administration (C₂₄), wherein the ratio of Cₘₐₓ₁ to Cₘᵢₙ₁ is less than about 2:1 or the ratio of Cₘₐₓ₂ to C₂₄ is less than about 2:1; or
f. a first maximum opioid plasma concentration (Cₘₐₓ₁) and a first minimum opioid plasma concentration (Cₘᵢₙ₁) between about 0 hours and about 12 hours after administration, a second opioid maximum plasma concentration (Cₘₐₓ₂), and an opioid plasma concentration at about 24 hours after administration (C₂₄), wherein the difference between the ratio of Cₘₐₓ₁ to Cₘᵢₙ₁ and the ratio of Cₘₐₓ₂ to C₂₄ is less than about 30%.

22. The oral dosage form of claim 1, wherein the dosage form, at steady state, provides a maximum opioid plasma concentration (Cₘₐₓ) and an opioid plasma concentration at about 24 hours after administration (C₂₄), wherein the ratio of Cₘₐₓ to C₂₄ is less than about 2:1.

23. The oral dosage form of claim 1, which at steady-state, provides a first Area Under the Curve (AUC₁) between 0 and about 12 hours and a second Area Under the Curve (AUC₂) between 12 hours and about 24 hours, wherein the difference between AUC₂ and AUC₁ is less than about 50%.

24. The oral dosage form of claim 1 wherein the sustained released material comprises a combination of an anionic alkyl salt and a pore-former.

25. The oral dosage form of claim 24 wherein the anionic alkyl salt is sodium lauryl sulfate and the pore-former is hydroxypropylcellulose.

26. A sustained-release oral dosage form comprising at least a first subunit and a second subunit, wherein:
a) each subunit comprises an opioid analgesic and a sustained-release material;
b) the dissolution rate of the first subunit measured by the USP Basket Method (Apparatus 1) at 50 rpm with 500 ml of 0.1 N HCl for 1 hour followed by 500 ml of pH 7.5 0.05 M phosphate buffer at 37° C. is greater than about 20% within about 6 hours;
c) the dissolution rate of the second subunit measured by the USP Paddle Method (Apparatus 2) at 100 rpm with 900 ml of pH 7.5 0.05 M phosphate buffer at 37° C. is at less than about 10% within about 6 hours.

27. The sustained-release oral dosage form of claim 26 wherein the opioid analgesic is morphine.
